(19) United States
(12) Patent Application Publication
(10) Pub. No.: US 2026/0123896 A1
(43) Pub. Date: May 7, 2026

(54) DIGITAL TOMOGRAPHIC IMAGE ACQUISITION DEVICE AND METHOD FOR CONTROLLING THE SAME

(71) Applicant: LG ELECTRONICS INC., Seoul (KR)

(72) Inventors: Taehyung KIM, Seoul (KR); Sungeun LEE, Seoul (KR); Byungkee LEE, Seoul (KR); Yongtae KIM, Seoul (KR); Youngjun JUNG, Seoul (KR); Dahea HAN, Seoul (KR)

(73) Assignee: LG ELECTRONICS INC., Seoul (KR)

(21) Appl. No.: 19/231,942

(22) Filed: Jun. 9, 2025

(30) Foreign Application Priority Data
Nov. 4, 2024 (WO) PCT/KR2024/017137

(51) Int. Cl. A61B 6/02; A61B 6/00; A61B 6/40; A61B 6/58

(52) U.S. Cl. CPC A61B 6/025; A61B 6/5205; A61B 6/54; A61B 6/4007; A61B 6/4452; A61B 6/582

(57) ABSTRACT

A digital tomographic image acquisition device is proposed according to an embodiment of the present disclosure. The digital tomographic image acquisition device includes: a generator configured to generate X-rays; and a detector configured to detect readout data corresponding to the generated X-rays. The detector may be configured to detect the readout data in a target readout area corresponding to a source area in which the X-rays generated by the generator are to be emitted. The target readout area may be set as a partial area of an entire readout area covered by the detector.

### CROSS-REFERENCE TO RELATED APPLICATION(S)

Pursuant to 35 U.S.C. § 119, this application claims the benefit of earlier filing date and right of priority to International Application No. PCT/KR2024/017137, filed on Nov. 4, 2024, the contents of which are all incorporated by reference herein in their entirety.

### BACKGROUND

### Technical Field

The present disclosure relates to an X-ray device and method for controlling the same, and more particularly, to a device for non-rotational tomographic imaging and method for controlling the same.

### Discussion of the Related Art

Digital tomosynthesis is an imaging diagnostic technology that combines the advantages of traditional X-ray technology and tomography. Digital tomosynthesis is primarily used in chest imaging and reconstructs three-dimensional (3D) images using X-ray images taken from multiple angles.

Digital tomosynthesis may be configured as follows.

X-ray imaging: An X-ray device captures multiple images from various angles around an examination area. In this case, the radiation exposure is similar to that of regular 2D X-ray imaging.

Image reconstruction: The captured images are processed by a processor or computer to reconstruct the captured images into 3D images. The reconstruction allows viewing cross-sections layer by layer, making it possible to clearly observe fine lesions that were not visible in conventional 2D X-ray images.

Tomographic image analysis: The 3D images may be analyzed by specific slices at various depths. Overlapping tissues are separated and displayed, thereby improving diagnostic accuracy.

As described above, digital tomosynthesis allows for more accurate detection of small lesions or abnormalities that are easily missed in 2D X-ray images, thereby making it advantageous for early diagnosis of various diseases. Additionally, digital tomosynthesis offers 3D images similar or comparable to computed tomography (CT) while maintaining lower radiation exposure. Further, by resolving the issue of overlapping tissues, digital tomosynthesis enables clearer visualization of lesions, thereby improving diagnostic accuracy.

### SUMMARY

Accordingly, the present disclosure is directed to a digital tomographic image acquisition device and method for controlling the same that substantially obviate one or more problems due to limitations and disadvantages of the related art.

The present disclosure aims to provide a digital tomographic image acquisition device or digital tomosynthesis device for faster digital tomosynthesis with reduced radiation exposure and control method therefor in the previously described digital tomosynthesis technology.

Additional advantages, objects, and features of the disclosure will be set forth in part in the description which follows and in part will become apparent to those having ordinary skill in the art upon examination of the following or may be learned from practice of the disclosure. The objectives and other advantages of the disclosure may be realized and attained by the structure particularly pointed out in the written description and claims hereof as well as the appended drawings.

To achieve these objects and other advantages and in accordance with the purpose of the disclosure, as embodied and broadly described herein, provided is a digital tomographic image acquisition device. The device includes: a generator configured to generate X-rays; and a detector configured to detect readout data corresponding to the generated X-rays. The detector may be configured to detect the readout data in a target readout area corresponding to a source area in which the X-rays generated by the generator are to be emitted. The target readout area may be set as a partial area of an entire readout area covered by the detector.

Additionally or alternatively, the target readout area may be configured to have a size determined based on position information on the source area corresponding to the target readout area or position information on the generator for the source area corresponding to the target readout area.

Additionally or alternatively, the generator may include a plurality of X-ray source units. The generator may be configured to sequentially emit the X-rays to source areas configured for each of the plurality of X-ray source units.

Additionally or alternatively, the generator may include a movable X-ray source unit. The movable X-ray source unit may be configured to sequentially emit the X-rays to a target source area configured for the movable X-ray source unit as the movable X-ray source unit moves. The target source area may be a part of an entire source area of the generator to emit the X-rays.

Additionally or alternatively, the detector may be configured to identify the target readout area and the generator or a position of the generator that emits the X-rays corresponding to the target readout area through pre-calibration.

Additionally or alternatively, the target readout area may be determined based on a radius of the target readout area or position information on a foot of a perpendicular dropped from an activated X-ray source unit of the generator to a plane on the detector that includes the target readout area. The radius of the target readout area may be determined based on a shortest distance between the generator and the detector and an X-ray emission angle of the generator.

Additionally or alternatively, the target readout area may be defined by a gate line range of the detector and a data line range of the detector. The gate line range may include a range on a first axis that falls within the radius of the target readout area, centered on a first-axis coordinate value of the position information, and the data line range may include a range on a second axis that falls within the radius of the target readout area, centered on a second-axis coordinate value of the position information.

Additionally or alternatively, the target readout area may be determined based on the position of the generator and the readout data to be detected corresponding to each of the position of the generator.

Additionally or alternatively, the gate line range and the data line range may be limited within a boundary of the entire readout area.

Additionally or alternatively, the detector may be configured to deactivate readout areas except for the target readout area.

In another aspect of the present disclosure, provided herein is a control method for a digital tomographic image acquisition device. The control method includes: generating X-rays through a generator; and detecting readout data in a target readout area corresponding to a source area in which the X-rays generated by the generator are to be emitted, through a detector. The target readout area may be set as a partial area of an entire readout area covered by the detector.

Additionally or alternatively, the target readout area may be configured to have a size determined based on position information on the source area corresponding to the target readout area or position information on the generator for the source area corresponding to the target readout area.

Additionally or alternatively, the generator may include a plurality of X-ray source units. The control method may include sequentially emitting the X-rays to source areas configured for each of the plurality of X-ray source units.

Additionally or alternatively, the generator may include a movable X-ray source unit. The control method may include sequentially emitting the X-rays to a target source area configured for the movable X-ray source unit while moving the movable X-ray source unit. The target source area may be a part of an entire source area of the generator configured to emit the X-rays.

Additionally or alternatively, the control method may include identifying the target readout area through pre-calibration.

Additionally or alternatively, the target readout area may be identified based on a radius of the target readout area or position information on a foot of a perpendicular dropped from an activated X-ray source unit of the generator to a plane on the detector that includes the target readout area. The radius of the target readout area may be determined based on a shortest distance between the generator and the detector and an X-ray emission angle of the generator.

Additionally or alternatively, the target readout area may be defined by a gate line range of the detector and a data line range of the detector. The gate line range may include a range on a first axis that falls within the radius of the target readout area, centered on a first-axis coordinate value of the position information, and the data line range may include a range on a second axis that falls within the radius of the target readout area, centered on a second-axis coordinate value of the position information.

Additionally or alternatively, the target readout area may be identified based on a position of the generator and the readout data to be detected corresponding to each of the position of the generator.

Additionally or alternatively, the gate line range and the data line range may be limited within a boundary of the entire readout area.

Additionally or alternatively, the control method may include deactivating readout areas except for the target readout area.

In a further aspect of the present disclosure, provided herein is a computer-readable medium storing code configured to cause a computer or a processor to execute the control method described above.

It will be understood by those skilled in the art that the above-described aspects of the present disclosure are merely part of various embodiments of the present disclosure, and various modifications and alternatives could be developed from the following technical features of the present disclosure.

The present disclosure has the following effects.

According to the present disclosure, during a process of obtaining multiple X-ray images, the images are captured in block units according to a non-rotational three-dimensional (3D) 3D X-ray imaging method, instead of capturing the entire area of a subject and detector at once. Accordingly, instead of detecting across the entire area of the detector, data may be read in the block units, thereby reducing the operation time of the detector.

The effects according to the present disclosure are not limited to what has been particularly described hereinabove, and any other effects not mentioned may be clearly understood by those skilled in the art to which the present disclosure pertains from the following detailed description.

### BRIEF DESCRIPTION OF THE DRAWINGS

The accompanying drawings, which are included to provide a further understanding of the disclosure and are incorporated in and constitute a part of this application, illustrate embodiment(s) of the disclosure and together with the description serve to explain the principle of the disclosure. In the drawings:

FIG. 1 illustrates a conceptual diagram of non-rotational X-ray imaging according to the present disclosure;

FIG. 2 illustrates a detector and readout area for X-ray image acquisition according to the present disclosure;

FIGS. 3 and 4 illustrate sequential activation of readout areas by a detector for X-ray image acquisition according to the present disclosure;

FIG. 5 is a diagram for explaining the types of X-ray generators in a digital tomographic image acquisition device or digital X-ray device according to the present disclosure;

FIG. 6 is a diagram for explaining a method of determining readout areas by a detector according to the present disclosure;

FIG. 7 is a flowchart illustrating a control method for a digital tomographic image acquisition device or digital X-ray device according to the present disclosure;

FIG. 8 illustrates an example of a digital tomographic image according to the present disclosure; and

FIG. 9 illustrates a block diagram of a digital tomographic image acquisition device or digital X-ray device according to the present disclosure.

### DETAILED DESCRIPTION

Hereinafter, embodiments of the present disclosure will be described in detail with reference to the attached drawings. In this specification, the same or equivalent components will be provided with the same reference numbers, and description thereof will not be repeated. The suffixes “module” and “unit” used for the components in the following description are assigned or used for convenience of description, and do not inherently have distinct meanings or roles. The suffixes are employed solely for ease of reference and should not be considered to convey unique distinctions in meaning or function. If it is deemed that detailed descriptions of the related art obscure the gist of the embodiments disclosed in this specification, the detailed descriptions will be omitted. It should be understood that the attached drawings are merely to provide better understanding of the embodiments disclosed herein and the technical concepts of the present disclosure are not limited to the attached drawings. Thus, the present disclosure should be construed to encompass all alterations, equivalents, and alternatives within the scope of the concepts and technologies disclosed in the present disclosure.

While terms such as “first,” “second,” and so on may be used to describe various components, but the aforementioned components are not limited by these terms. The above terms are used only to distinguish one component from another.

When a component is mentioned to be “connected” or “coupled” to another component, it may be directly connected or coupled to the other component, but it should be understood that there could also be other components in between. On the other hand, when a component is mentioned to be “directly connected” or “directly coupled” to another component, it should be understood that there are no other components in between.

Unless singular expressions clearly indicate otherwise in context, the singular expressions encompass plural expressions.

In the present disclosure, terms such as “comprises” or “includes” are intended to indicate the presence of features, numbers, steps, operations, components, parts, or combinations thereof as specified in the specification, rather than to preclude the presence or possibility of one or more other features, numbers, steps, operations, components, parts, or combinations thereof.

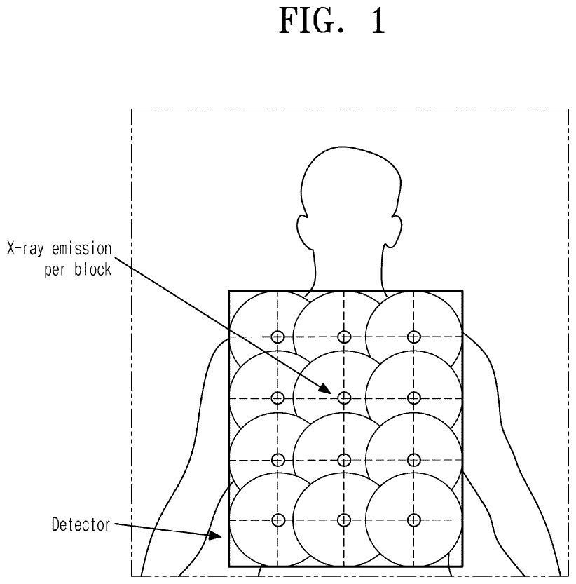

FIG. 1 illustrates a conceptual diagram of non-rotational X-ray imaging according to the present disclosure.

A device for acquiring digital tomographic images (or a device for capturing digital tomographic images) (hereinafter referred to as a digital tomographic image acquisition device) obtains multiple X-ray images and reconstructs the multiple X-ray images to acquire three-dimensional (3D) tomographic images.

In this process, capturing multiple images requires a time ranging from several seconds to tens of seconds. In particular, as the resolution of a detector increases, the time required to read sensing values from each cell of the detector also increases.

According to the present disclosure, in a non-rotational 3D system, when X-rays pass through a subject and are transmitted to the detector, an X-ray generator is controlled to emit, pass through the subject, and transmit X-rays in block units, unlike general tomographic image acquisition devices. Accordingly, the present disclosure controls the detector to read data in the block units.

According to the present disclosure, the detector reads sensing data only from a target readout area, where images are projected, rather than an entire readout area. This reduces the detector operation time and enables high-speed operation.

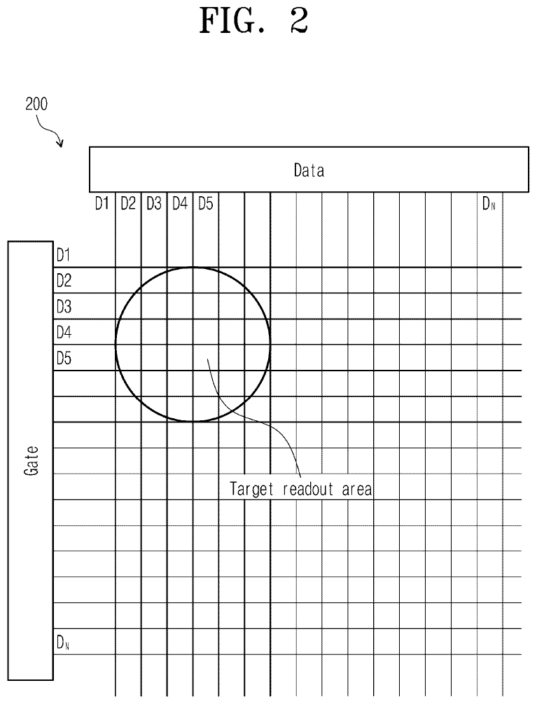

FIG. 2 illustrates a detector and readout area for X-ray image acquisition according to the present disclosure.

The detector is configured to read sensing data only from a target readout area corresponding to an area where X-rays are actually emitted by a generator (hereinafter referred to as a “source area”), rather than reading the entire gate and data lines of the readout area of the detector. As a result, the imaging or operation time of the non-rotational tomographic image acquisition device may be reduced. The larger the size of the detector, the greater the effect.

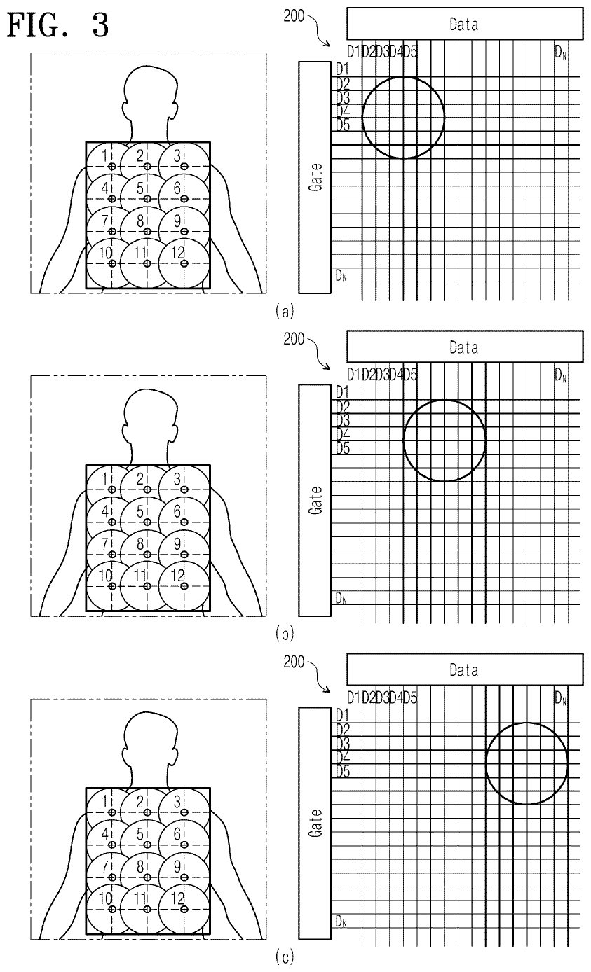

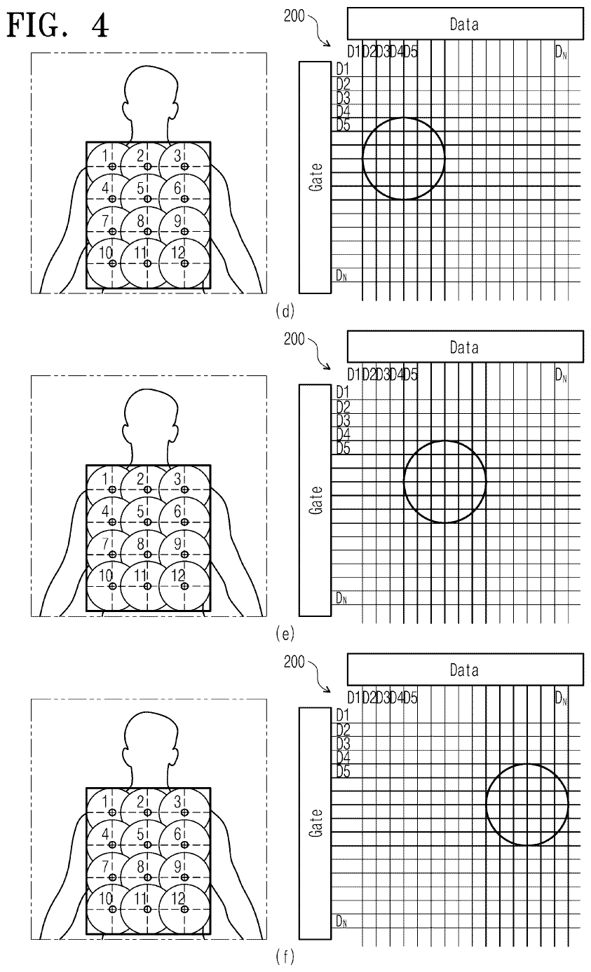

FIGS. 3 and 4 illustrate sequential activation of readout areas by a detector for X-ray image acquisition according to the present disclosure.

In (a) to (f) of FIGS. 3 and 4, target readout areas for blocks 1 to 6 out of a total of 12 blocks (source areas or target readout areas) are sequentially illustrated.

However, as will be described later, the source areas from the perspective of the generator and the target readout areas from the perspective of the detector may not be entirely identical. Preferably, the target readout areas may be configured to include the source areas.

Additionally, the relationship between target readout areas is not specifically defined. In other words, a first target readout area corresponding to a first time point or first source area and a second target readout area corresponding to a second time point or second source area may partially overlap. That is, the first target readout area and the second target readout area may be configured to include at least different areas.

Accordingly, the detector needs to have information (e.g., position or coordinate information) about the source area activated at each readout time. The detector activates the target readout area corresponding to the source area and performs data readout within the activated target readout area.

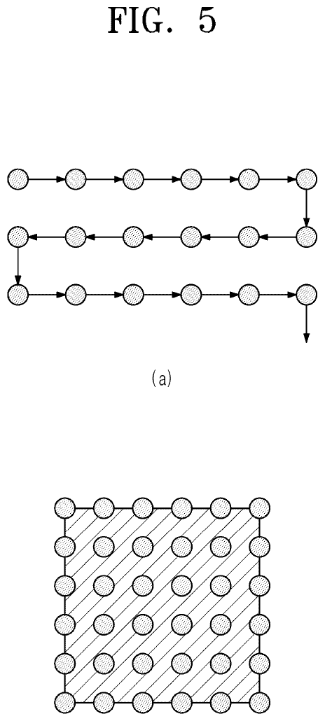

FIG. 5 is a diagram for explaining the types of X-ray generators in a digital tomographic image acquisition device or digital X-ray device according to the present disclosure.

Specifically, (a) of FIG. 5 illustrates the sequential movement of a source area when a generator is configured with a single X-ray source (or emission) unit and is configured to be movable. Referring to (a) of FIG. 5, the source area is configured starting from the top-left corner and moves in a zigzag pattern toward the right, and the generator moves accordingly. The source area for a movable generator or X-ray source unit shown in (a) of FIG. 5 is referred to as a target source area.

Specifically, (b) of FIG. 5 illustrates a source area when a generator is configured with multiple X-ray sources (or emission) units arranged in a two-dimensional configuration. Each point in (b) of FIG. 5 may represent a single X-ray source unit. In the previous explanation, it has been described that only one of multiple X-ray source units is activated (i.e., emits X-rays) at a given time point (during an X-ray emission cycle or readout cycle). However, the present disclosure is not limited thereto. Two or more X-ray source units may be activated simultaneously, and accordingly, two or more target readout areas may be activated simultaneously. In other words, a detector may activate multiple target readout areas and read sensing data in parallel.

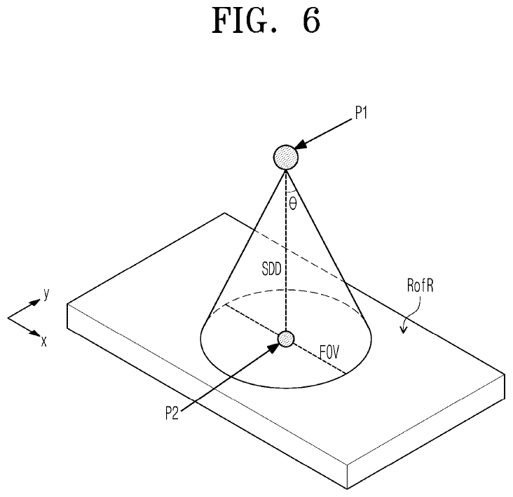

FIG. 6 is a diagram for explaining a method of determining readout areas by a detector according to the present disclosure.

FIG. 6 shows determination of a target readout area based on the specifications (or parameters, etc.) of a detector or generator in a digital tomographic image acquisition device. It is assumed that the target readout area is a circle on a plane, the diameter of the target readout area is referred to as a field of view (FOV).

The source unit (i.e., X-ray emission source) of a generator 100 is denoted as P1, and the foot of the perpendicular dropped from P1 to a plane RofR that includes the readout area is denoted as P2.

In this case, the FOV may be determined as follows.

$$F O V = 2 * SDD * tan ( θ )$$

Here, SDD represents the minimum distance between the generator 100 and

a detector 200, and theta (θ) represents the X-ray emission angle of the generator 100. For reference, theta (θ) is determined by the anode angle of an X-ray tube and the design of a collimator.

Using the FOV, the range of gate lines and data lines of the detector 200 may be determined as follows.

$$Starting position of gate line = ( y - axis coordinate or position of P 2 - h - F O V )$$

$$Ending position of gate line = ( y - axis coordinate or position of P 2 + h - F O V )$$

$$Here , h - F O V = F O V / 2.$$

$$Starting position of data line = ( x - axis coordinate or position of P 2 - h - F O V )$$

$$Ending position of data line = ( x - axis coordinate or position of P 2 + h - F O V )$$

However, since the FOV value is in the dimension of length, performing coordinate transformation allows obtaining information on the range of the gate lines or data lines.

The coordinate transformation of the FOV may be achieved by dividing the FOV by the pixel size of the detector.

For example, in the case of a 17-inch detector (43 cm×43 cm, 3072×3072 pixels, and pixel size=0.14 mm) and SDD=45 cm, if the position of the source unit of the generator is at coordinates 1536 and 1536 and the X-ray emission angle is 12 degrees, the FOV, the coordinate transformation of h-FOV, and the range of the gate lines and data lines may be determined as follows.

$$F O V = 2 * 45 * tan ( 12 ) = 19.13 cm , h - F O V = 9.56 cm , and h - F O V coordinate transformation = 95.6 \div 0.14 = 683 \qquad ( 1 )$$

$$Start of gate line = 1536 - 683 = 853 line \qquad ( 2 )$$

$$End of gate line = 153 \; 6 + 683 = 2219 line \qquad ( 3 )$$

$$Start of data line = 1536 - 683 = 853 line \qquad ( 4 )$$

$$End of data line = 153 \; 6 + 683 = 2219 line \qquad ( 5 )$$

Referring to (a) or (b) of FIG. 5, since P1 may have multiple positions, the detector may acquire the position or coordinates of P1 (or P2) and determine the range of gate lines and data lines based thereon. The detector may determine the target readout area based on the acquired range of gate lines and data lines.

The detector activates the target readout area corresponding to the active source area of the generator, which means activating the data lines and gate lines as described above. By reading sensing data from the activated target readout area, the detector enables faster operation compared to activating all readout areas, i.e., all data lines and gate lines.

The range of the gate lines or data lines of the detector may be obtained through measurement rather than based on the specifications (or parameters, etc.) of the detector or generator in the digital tomographic image acquisition device as shown in FIG. 6.

A non-rotational tomographic image acquisition device may obtain information on the position of the source unit of a generator relative to a detector through geometry calibration in advance. This will be briefly explained. When the generator emits X-rays from predetermined positions, the FOV projected onto the detector is calibrated in advance to acquire detector coordinate values.

For all positions where the generator emits X-rays, the detector obtains the coordinate values of an area where the X-rays are projected. Accordingly, the positions where the generator emits the X-rays and the position information (i.e., coordinate information) on the area where the X-rays are projected on the detector may be acquired in advance. Through this procedure, it is possible to obtain information about the range of gate lines or data lines of the detector to be activated or the target readout area, which corresponds to the generator or the source unit of the generator.

When the X-rays are emitted based on the information, the detector reads data by activating related gate lines and data lines. For areas where the X-rays do not reach (i.e., areas outside the range of gate lines and data lines), the gate lines and data lines are deactivated.

According to another method, the target readout area may be determined as the larger area between the areas determined by the two methods described above. That is, the target readout area may be determined as the larger area between the target readout area based on the specifications (or parameters, etc.) of the detector or generator in the digital tomographic image acquisition device and the target readout area based on the results of the X-rays or readout data based thereon.

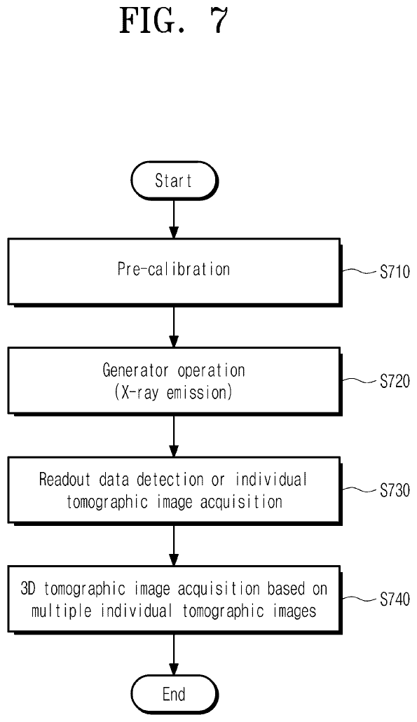

FIG. 7 is a flowchart illustrating a control method for a digital tomographic image acquisition device or digital X-ray device according to the present disclosure. The flowchart shown in FIG. 7 may be performed by the digital tomographic image acquisition device or the processor thereof. However, the present disclosure is not limited thereto. Hereinafter, for simplicity of explanation, the flowchart will be described as being performed by a device 10.

The device 10 may perform pre-calibration (S710). The pre-calibration may include the geometry calibration described above. The calibration is a procedure to obtain the matching relationship between a generator or a source unit of the generator and a readout area or target readout area. The matching relationship may include the relationship between the generator or source unit (or position thereof) and a tomographic image obtained based on data resulting from X-rays emitted by the generator or source unit.

Thus, information on the position of the generator or source unit (e.g., coordinate information) may be used when obtaining or reconstructing a 3D tomographic image based on multiple two-dimensional (2D) tomographic images. The details thereof will be described later.

Accordingly, the device 10 may configure the target readout area based on a source area.

The device 10 may emit X-rays through the generator (S720). The X-ray emission of the generator may be performed as illustrated in FIG. 4, depending on the type of generator.

The device 10 may detect readout data from the target readout area using a detector and, based on the readout data, acquire individual tomographic images (S730). Each tomographic image may include a single image corresponding to one source unit of the generator or information on the position of the source unit. For example, when a 2*2 source unit array is used as the generator, the individual tomographic images acquired at the corresponding step may total four tomographic images.

The device 10 may acquire a single 3D tomographic image using multiple individual tomographic images (S740). In this case, the matching relationship between the previously acquired individual tomographic images and the generator or source unit that emits the corresponding X-rays may be used. The positions of multiple source units are different, and thus, the data or tomographic images acquired in the target readout area may have different depths (distance between the generator and the detector). Based on the differences, a 3D tomographic image may be obtained.

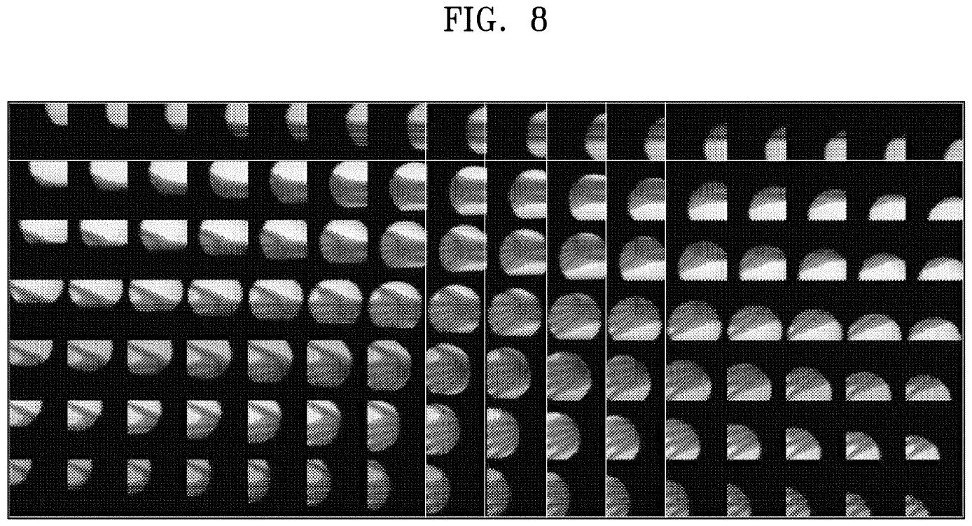

FIG. 8 illustrates an example of a digital tomographic image according to the present disclosure.

In FIG. 8, a total of 16*7 tomographic images are shown. The multiple tomographic images in FIG. 8 represent images sequentially acquired by a detector 200 and are arranged in time order, starting from the top-left, prioritizing the column direction first and then the row direction, proceeding downward and to the right. The time order is explained based on a generator 100. The generator 100 emits or generates X-rays starting from the top-right (in a source area or readout area), moving first in the row direction and then in the column direction, proceeding to the left and downward.

As illustrated, in each tomographic image, a portion where valid data is detected (subject detection) is shown in white or shades of gray, while a portion corresponding to invalid data (no subject detection) is shown in black. A target readout area corresponds to the portion where valid data is detected and, preferably, may include at least the portion where valid data is detected.

For convenience of explanation, portions shown in colors other than black in each tomographic image in FIG. 8 will be referred to as the target readout area.

As illustrated, the size of the target readout area varies for each tomographic image. The target readout area is associated with the position of the activated X-ray generator 100 or the source area of the generator 100. The closer the activated X-ray generator 100 or the source area thereof is to the center of an entire source area, the larger the corresponding target readout area may be set.

Additionally, the target readout area may be limited by the boundary of an entire readout area. For example, in the top-left tomographic image of FIG. 7, the target readout area is limited by the boundary line defining the top-right corner of the entire readout area (individual image area or equivalent thereof).

Depending on the size and position of the source area or target readout area corresponding thereto, the target readout area to be activated within the entire readout area may vary. Since the detector 200 performs readout only for a portion of the entire readout area, the readout speed may be faster than performing readout for the entire readout area.

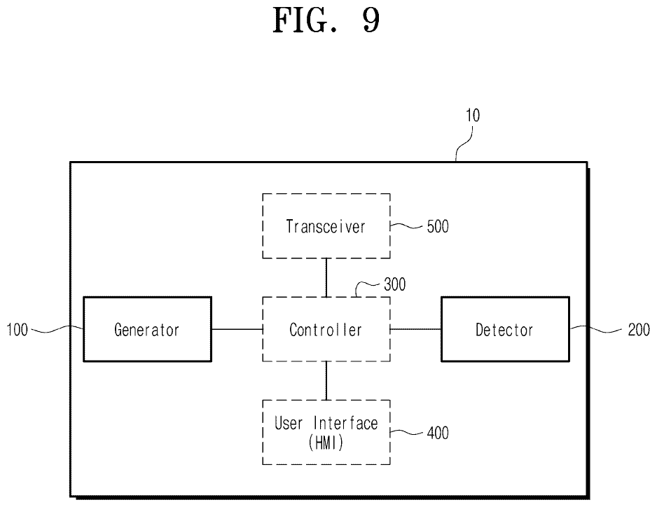

FIG. 9 illustrates a block diagram of a digital tomographic image acquisition device 10 or digital X-ray device according to the present disclosure.

The digital tomographic image acquisition device 10 may include: a generator 100 configured to generate and emit X-rays; and a detector 200 configured to detect X-rays that pass through a subject or readout data corresponding to the X-rays that pass through the subject. Each of the generator 100 and the detector 200 may have a controller embedded therein to control operations thereof.

Additionally, the digital tomographic image acquisition device 10 may include a controller 300. The controller 300 may be included when the generator 100 or detector 200 does not have individual controllers. The controller 300 may control the emission of X-rays from the generator 100 or the readout of sensor data from the detector 200. Furthermore, the controller 300 may control a user interface 400 or a transceiver 500, which will be described later.

Additionally, the digital tomographic image acquisition device 10 may include a user interface (human-machine interface (HMI)) 400 for outputting acquired tomographic images or 3D tomographic images obtained from tomographic images. The user interface (HMI) may include a display.

Additionally, the digital tomographic image acquisition device 10 may include the transceiver 500 for transmitting the acquired tomographic images or the 3D tomographic images obtained from the tomographic images to other devices or systems. Furthermore, the transceiver 500 may be configured to receive information on logic or algorithms for tomographic image acquisition or calibration, or information on logic or algorithms that indicate an order or method for X-ray emission of the generator 100 or the source unit of the generator 100.

Hereinafter, it is described that the generator 100 and the detector 200 perform operations according to the present disclosure. However, the operations according to the present disclosure may instead be performed by the controller 300.

The generator 100 may be configured to emit X-rays in a predetermined order or from predetermined positions.

The detector 200 may be configured to detect readout data in a target readout area that corresponds to the generator 100 or a source area where the X-rays generated by the generator 100 are to be emitted. In this case, the target readout area may be set as a part of the entire readout area covered by the detector 200.

The detector 200 may be configured to deactivate readout for areas except for the target readout area.

The target readout area may be configured to have a size determined based on the source area of the generator 100 corresponding to the target readout area or information on the position of the generator 100 for the source area thereof corresponding to the target readout area.

The generator 100 includes multiple X-ray source units and may be configured to sequentially emit X-rays to source areas configured for each of the multiple X-ray source units.

The generator 100 includes a movable X-ray source unit and may be configured to sequentially emit X-rays to a target source area configured for the movable X-ray source unit as the movable X-ray source unit moves. The target source area may be a part of the entire source area where the generator 100 is capable of emitting X-rays.

The detector 200 may be configured to identify the target readout area and the generator or the position of the generator that emits the corresponding X-rays through pre-calibration.

The target readout area is determined based on the radius of the target readout area, information on the position of the generator, or information on the position of the foot of the perpendicular dropped from an activated X-ray source unit of the generator to a plane on the detector that includes the target readout area. The radius of the target readout area may be determined based on the shortest distance between the generator and the detector and the X-ray emission angle of the generator. This method is referred to as a first method for determining the target readout area.

The target readout area may be determined based on information on the position of the generator and readout data detected based on information on each position of the generator. This method is referred to as a second method for determining the target readout area.

Alternatively, the target readout area may be determined as the larger area between the target readout areas determined by the first method or the second method.

The target readout area is defined by the ranges of gate and data lines of the detector. The gate line range includes a range on a first axis that falls within the radius of the target readout area, centered on a first-axis coordinate value of information on the position of the foot of the perpendicular. The data line range includes a range on a second axis that falls within the radius of the target readout area, centered on a second-axis coordinate value of information on the position of the foot of the perpendicular. Meanwhile, the gate line range and data line range may be limited within the boundary of the entire readout area of the detector 200.

Even if not explained with reference to FIG. 9, the digital tomographic image acquisition device 10 of the present disclosure may perform the operations described above with reference to FIGS. 2 to 8 according to the present disclosure.

In another aspect of the present disclosure, the above-described proposals or inventive operations may also be provided as code capable of being implemented, performed, or executed by a “computer” (i.e., a comprehensive concept including a system-on-chip (SoC) or a processor (or microprocessor), a computer-readable storage medium including the aforementioned code, or a computer program product. The scope of the present disclosure may be extended to the code, the computer-readable storage medium including the code, or the computer program product.

The exemplary embodiments of the present disclosure have been provided to enable those skilled in the art related to the present disclosure to implement and practice the present disclosure. Although the above description has been provided with reference to the exemplary embodiments of the present disclosure, it will be understood by those skilled in the art that the present disclosure as set forth in the claims below may be modified and varied in various ways. Therefore, the present disclosure is intended to provide the broadest scope consistent with the principles and novel features disclosed herein, rather than being limited to the embodiments disclosed herein.

What is claimed is:

1. A digital tomographic image acquisition device comprising:
a generator configured to generate X-rays; and
a detector configured to detect readout data corresponding to the generated X-rays,
wherein the detector is configured to detect the readout data in a target readout area corresponding to a source area in which the X-rays generated by the generator are to be emitted, and
wherein the target readout area is set as a partial area of an entire readout area covered by the detector.

2. The digital tomographic image acquisition device of claim 1, wherein the target readout area is configured to have a size determined based on position information on the source area corresponding to the target readout area or position information on the generator for the source area corresponding to the target readout area.

3. The digital tomographic image acquisition device of claim 1, wherein the generator comprises a plurality of X-ray source units, and
wherein the generator is configured to sequentially emit the X-rays to source areas configured for each of the plurality of X-ray source units.

4. The digital tomographic image acquisition device of claim 1, wherein the generator comprises a movable X-ray source unit,
wherein the movable X-ray source unit is configured to sequentially emit the X-rays to a target source area configured for the movable X-ray source unit as the movable X-ray source unit moves, and
wherein the target source area is a part of an entire source area of the generator configured to emit the X-rays.

5. The digital tomographic image acquisition device of claim 1, wherein the detector is configured to identify the target readout area and the generator or a position of the generator that emits the X-rays corresponding to the target readout area through pre-calibration.

6. The digital tomographic image acquisition device of claim 1, wherein the target readout area is determined based on a radius of the target readout area or position information on a foot of a perpendicular dropped from an activated X-ray source unit of the generator to a plane on the detector that includes the target readout area, and
wherein the radius of the target readout area is determined based on a shortest distance between the generator and the detector and an X-ray emission angle of the generator.

7. The digital tomographic image acquisition device of claim 6, wherein the target readout area is defined by a gate line range of the detector and a data line range of the detector,
wherein the gate line range includes a range on a first axis that falls within the radius of the target readout area, centered on a first-axis coordinate value of the position information, and
wherein the data line range includes a range on a second axis that falls within the radius of the target readout area, centered on a second-axis coordinate value of the position information.

8. The digital tomographic image acquisition device of claim 5, wherein the target readout area is determined based on the position of the generator and the readout data to be detected corresponding to each of the position of the generator.

9. The digital tomographic image acquisition device of claim 7, wherein the gate line range and the data line range are limited within a boundary of the entire readout area.

10. The digital tomographic image acquisition device of claim 1, wherein the detector is configured to deactivate readout areas except for the target readout area.

11. A control method for a digital tomographic image acquisition device, the control method comprising:
generating X-rays through a generator; and
detecting readout data in a target readout area corresponding to a source area in which the X-rays generated by the generator are to be emitted, through a detector,
wherein the target readout area is set as a partial area of an entire readout area covered by the detector.

12. The control method of claim 11, wherein the target readout area is configured to have a size determined based on position information on the source area corresponding to the target readout area or position information on the generator for the source area corresponding to the target readout area.

13. The control method of claim 11, wherein the generator comprises a plurality of X-ray source units, and
wherein the method comprises sequentially emitting the X-rays to source areas configured for each of the plurality of X-ray source units.

14. The control method of claim 11, wherein the generator comprises a movable X-ray source unit,
wherein the method comprises sequentially emitting the X-rays to a target source area configured for the movable X-ray source unit while moving the movable X-ray source unit, and
wherein the target source area is a part of an entire source area of the generator configured to emit the X-rays.

15. The control method of claim 11, comprising identifying the target readout area through pre-calibration.

16. The control method of claim 11, wherein the target readout area is identified based on a radius of the target readout area or position information on a foot of a perpendicular dropped from an activated X-ray source unit of the generator to a plane on the detector that includes the target readout area, and
wherein the radius of the target readout area is determined based on a shortest distance between the generator and the detector and an X-ray emission angle of the generator.

17. The control method of claim 16, wherein the target readout area is defined by a gate line range of the detector and a data line range of the detector,
wherein the gate line range includes a range on a first axis that falls within the radius of the target readout area, centered on a first-axis coordinate value of the position information, and
wherein the data line range includes a range on a second axis that falls within the radius of the target readout area, centered on a second-axis coordinate value of the position information.

18. The control method of claim 15, wherein the target readout area is identified based on a position of the generator and the readout data to be detected corresponding to each of the position of the generator.

19. The control method of claim 17, wherein the gate line range and the data line range are limited within a boundary of the entire readout area.

20. A computer-readable medium storing code configured to cause a computer or a processor to execute the method according to claim 11.
